(19) United States
(12) Patent Application Publication
(10) Pub. No.: US 2014/0213937 A1
(43) Pub. Date: Jul. 31, 2014

(54) SYSTEMS AND METHODS TO MONITOR AND QUANTIFY PHYSIOLOGICAL STAGES

(71) Applicants: Matt T. Bianchi, Somerville, MA (US); Thomas Lipoma, Boston, MA (US); Carson J. Darling, Boston, MA (US); Pablo J. Bello, Boston, MA (US)

(72) Inventors: Matt T. Bianchi, Somerville, MA (US); Thomas Lipoma, Boston, MA (US); Carson J. Darling, Boston, MA (US); Pablo J. Bello, Boston, MA (US)

(73) Assignee: THE GENERAL HOSPITAL CORPORATION, Boston, MA (US)

(21) Appl. No.: 13/982,560

(22) PCT Filed: Jul. 7, 2011
(86) PCT No.: PCT/US11/43173

§ 371 Date: Mar. 5, 2014

Related U.S. Application Data
(60) Provisional application No. 61/452,941, filed on Mar. 15, 2011.
(60) Provisional application No. 61/442,937, filed on Feb. 15, 2011.

(51) Int. Cl. A61B 5/00; A61B 5/113

(52) U.S. Cl. CPC A61B 5/4815; A61B 5/1135
USPC 600/595; 600/534

(57) ABSTRACT

Systems and methods detect respiration of a subject, process respiration data, and based on the processed respiration data, perform extended monitoring. A monitoring system is configured with sensors that can be worn by the subject to provide the respiration data. The respiration data is processes to create a stability index. The stability index is used to, for example, determine sleep stages.

### CROSS-REFERENCE TO RELATED APPLICATIONS

This application claims the benefit of U.S. Provisional Patent Application Ser. No. 61/442,937, filed Feb. 15, 2011, and entitled “A SLEEP-MEASURING SHIRT BASED ON EXPANSION AND RESPIRATORY PATTERNS,” which is hereby incorporated by reference.

This application also claims the benefit of U.S. Provisional Patent Application Ser. No. 61/452,941, filed Mar. 15, 2011, and entitled “A SLEEP-MEASURING SHIRT BASED ON EXPANSION AND RESPIRATORY PATTERNS,” which is hereby incorporated by reference.

### STATEMENT REGARDING FEDERALLY SPONSORED RESEARCH OR DEVELOPMENT

This invention was made with government support under DAMD 17-02-2-0006 awarded by U.S. Army Medical Acquisition Activity (USAMRAA). The government has certain rights in the invention.

### BACKGROUND OF THE INVENTION

The subject matter disclosed herein relates generally to systems and methods that monitor physiological states, such as in sleep, and, more particularly, that monitor and quantify sleep stages and/or sleep fragmentation.

Many millions of people around the world suffer from sleep disorders, such as insomnia, on a regular basis. Unfortunately for these individuals, emphasis has been placed on treatment rather than prevention. Sleep studies, the gold standard for diagnosing sleep disorders, are only used in medical cases potentially involving other, more serious conditions, such as sleep apnea or periodic limb movement disorder. This leaves all those who do not suffer from either of these two diseases with little to no feedback on their overall sleep health from a medical standpoint.

Sleep staging (or sleep study) procedures vary, but most employ monitoring respiration, electroencephalography (EEG), and electromyography (EMG) data. Some sleep studies are inherently expensive because of their employment of polysomnography (PSG), which, while critical for detection of various sleep disorders, is extremely costly. A polysomnogram (also PSG) monitors many body functions including brain (EEG), eye movements (EOG), muscle activity or skeletal muscle activation (EMG), heart rhythm (ECG), breathing functions, and peripheral pulse oximetry during sleep. Although in 2001 over a million PSGs were performed, and in 2008 over four million PSGs were performed in the United States alone, a lack of budget-friendly alternatives to sleep studies for accurate sleep health monitoring leaves many people with insomnia and other sleep disorders undiagnosed.

Laboratory testing is also disruptive to the regular sleep routines of the average subject. Subjects are asked to sleep away from their homes, in a different bed, and wearing an array of electrodes and bands. This makes for a less-than-perfect test, as comfort could potentially directly affect sleep quality.

Except in rare cases, PSGs are conducted inside a sleep laboratory. Subjects are typically instrumented with over twenty sensors—over a dozen on the head to measure EEG and muscle movement around the eye, two for air flow in and around the mouth and nose, one on the leg for EMG, two respiration belts, and three electrodes for measuring heart rate. These form a large bundle that is not only uncomfortable, but restricts the motion of the subject. A paid technician monitors PSG subjects at all times, and once the night is over, the technician examines the data and, using the various signals, creates a hypnogram showing a summary of sleep stages. Based on its very nature, the technician scoring is subjective, and depending on the particular sleep scoring metric, the rate of agreement among expert scorers for the staging results is 70-80 percent.

Alternative approaches to the PSG are available, yet none make significant improvements to the issues with PSG due to cost, physical constraints, and/or quality of data provided. Some at-home devices have entered the medical sleep monitoring industry, but most are still too uncomfortable, unreliable, or expensive to be routinely employed for medical diagnosis.

While PSG is the current standard for sleep staging, respiration has been found to be a good marker for wakefulness and rapid eye movement (REM) stages, making it also easy to detect whether a person is in light or deep sleep (non-REM stages). One known method for determining respiration includes using wireless, range-finding devices that detect changes in the distance to the subject's chest. However, this method requires special apparatuses to be installed and is dependent on the positioning of the subject within the range detector's field of view. Other methods use pressure sensors placed under the subject's mattress. Systems of these types require the subject to be in a supine position and any changes to this position can cause the systems to fail to record respiration data.

Other options use an adhesive EKG device that is approved for home monitoring in sleep apnea subjects. Fluctuations in the EKG signal are analyzed to stage sleep. This option is only capable of providing an indirect metric of respiratory patterns, and includes no actual respiration monitor. Because of the nature of the signal processing problem to combine EKG with respiration, it requires an extensive windows of analysis, for example, on the order of at least an eight minute window.

Another device is advertised as a device for first responders to measure vital signs using wireless transmission. The device only provides a rolling average respiration rate, not a respiration waveform. Because a continuous waveform over a period of time is required to effectively score sleep, this device is unable score sleep based on an average rate of respiration.

U.S. Pat. Nos. 7,150,718 and 7,427,270 describe systems that use a respiration signal to determine sleep state. The systems use variations of signal amplitude in the determination of sleep state. These systems have drawbacks because amplitude can change between subjects, and can change over the night if the subject is laying supine versus lateral position, for example.

It has been estimated that only a small fraction (less than 20 percent) of the population with sleep disorders even approach their caregivers about the issue. In addition, because the doctor's decision-making employs the use of a subjective sleep history given by the patient, there is a danger of misdiagnosis. Finally, even if a subject is diagnosed with the correct disorder and provided treatment, there are few options for patients to monitor their improvement and see the effect of the treatment.

It would, therefore, be desirable to provide systems and methods that are suitable for monitoring and analysis of sleep data to quantify sleep stages and/or sleep fragmentation in a manner consistent with clinical sleep staging, but that is not encumbered by complex and expensive mechanisms that can interfere with actual sleep and are not generally suitable for long-term use.

### BRIEF DESCRIPTION OF THE INVENTION

The present invention overcomes the aforementioned problems by providing systems and methods that accurately monitor sleep health while allowing the subject to sleep virtually anywhere he or she normally does. A suitable sleeping component, such as a shirt, may utilize embedded sensors to directly measure respiration of the subject throughout the night. Associated software analyzes the respiration data and uses relative frequency energies and bandwidth in a time-frequency analysis to determine the sleep stages. Consistent with the above comments, in some embodiments, the systems and methods comprise at least one co-planar pad capacitor sensor connected to an electronics package that allows for data gathering and transmission. The data can then be analyzed to determine sleep staging, including arousals from sleep.

In accordance with one aspect of the invention a system for collecting and analyzing sleep of a subject is disclosed that includes a sensor configured to detect a characteristic of respiration of the subject and a collection circuit connected to the sensor to receive an indication of the characteristic of respiration detected by the sensor. The system also includes a processing system configured to communicate with the collection circuit and generate a time series of respiration data from the indication of the characteristic of respiration over time and process the time series of respiration data to identify a subject-specific mechanism for identifying at least one of inhalation and exhalation. The processing system is further configured to analyze respiration volatility using the time series of respiration data and subject-specific mechanism for identifying at least one of inhalation and exhalation and, using the respiration volatility, generate a stability index relating fundamental characteristics of the time series of respiration data to metrics of stability. Based on the stability index, the processing system is configured to perform a sleep staging analysis to generate a report on the sleep of the subject over time.

In accordance with another aspect of the invention, a system for performing sleep staging of a subject is disclosed that includes a capacitive sensor that provides sensor capacitance data configured to vary over time based on breathing periods of the subject. The system also includes a circuit configured to evaluate the sensor capacitance data against a known reference capacitor and a computer-readable code embodied in or on a computer usable medium. The computer-readable code is configured to cause a computer to perform the steps of processing the capacitance data to identify breathing periods of the subject, creating a matrix that indicates the start of each breath, and running the matrix data through at least one fast Fourier transform. The computer-readable code is configure to cause the computer to perform the steps of creating a stability index from data points generated by the at least one fast Fourier transform, creating a time index based on a relative change in the stability index over time, and determining sleep stages using the stability index and the time index.

In accordance with yet another aspect of the invention, a method for analyzing sleep stages is disclosed that includes detecting respiration of a subject, the subject wearing a monitoring system that includes at least one capacitive sensor that provides respiration data. The method also includes processing the respiration data to identify breathing periods of the subject, creating a matrix that indicates the start of each breath, and running the matrix data through at least one fast Fourier transform. The method further includes creating a stability index from data points generated by the at least one fast Fourier transform, creating a time index based on a relative change in the stability index over time, and determining sleep stages using the stability index and the time index.

In accordance with still another aspect of the invention, a method for collecting and analyzing sleep of a subject is disclosed that includes detecting a characteristic of respiration of the subject over time and identifying, from the characteristic of respiration of the subject over time, a subject-specific mechanism for identifying at least one of inhalation and exhalation in the characteristic of respiration of the subject over time. The method also includes generating, based on identification of the at least one of inhalation and exhalation in the characteristic of respiration of the subject over time, a stability index relating fundamental characteristics of the time series of respiration data to metrics of stability and, from the stability index, generating a time index based on a relative change in the stability index over time. The method further includes identifying, based on the stability index and the time index, states of sleep of the subject and generating a report on the sleep of the subject over time indicating the identified states of sleep of the subject.

In accordance with yet another aspect of the invention, a sensor system is disclosed that includes a substrate having an elasticity. The sensor system also includes a first conductive pad engaged with the substrate and extending along a length and a width to form a plane and a second conductive pad engaged with the substrate, extending along a length and a width to form a plane, and arranged proximate to the first conductive plane. The sensor system further includes a dielectric gap formed between the first and second conductive pads such that at least one of a length and a width of the first conductive pad extends substantially in parallel to at least one of a length and a width of the second conductive pad. Also, the sensor system includes a processor coupled to the first conductive pad and the second conductive pad and configured to monitor a relative capacitance between the first conductive pad and the second conductive pad as the dielectric gap is varied in response to movement imparted to the substrate and generate an indication of a characteristic of the movement imparted to the substrate.

To the accomplishment of the foregoing and related ends, the embodiments, then, comprise the features hereinafter fully described. The following description and the annexed drawings set forth in detail certain illustrative aspects of the invention. However, these aspects are indicative of but a few of the various ways in which the principles of the invention can be employed. Other aspects, advantages and features of the invention will become apparent from the following detailed description of the invention when considered in conjunction with the drawings.

### BRIEF DESCRIPTION OF THE DRAWINGS

The embodiments will hereafter be described with reference to the accompanying drawings, wherein like reference numerals denote like elements, and:

FIG. 1A is a schematic diagram of a shirt including sensors for gathering respiration data according to embodiments of the invention;

FIG. 2 is a schematic diagram of a sensor as shown in FIG. 1A;

FIG. 3 is a graph showing a comparison of theoretical and actual capacitance values;

FIG. 4 is a flow chart setting forth the steps of a method that may be performed in accordance with embodiments of the invention;

FIG. 5 is a graph showing data points from a fast Fourier transform using samples of respiration data;

FIG. 6 is a flow chart setting forth the steps of a method that may be performed in accordance with embodiments of the invention;

FIG. 7 is a graph showing a sleep heat map usable for visualization of sleep stages;

FIG. 8 is a graph showing a frequency stability graph usable for visualization of sleep stages; and

FIG. 9 is a graph showing a sample of respiration data gathered from an embodiment of the shirt shown in FIG. 1.

### DETAILED DESCRIPTION OF THE INVENTION

Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures. The figures depict selected embodiments and are not intended to limit the scope of embodiments of the invention. Skilled artisans will recognize the examples provided herein have many useful alternatives and fall within the scope of embodiments of the invention.

The following description refers to elements or features being “connected” or “coupled” together. As used herein, unless expressly stated otherwise, “connected” means that one element/feature is directly or indirectly connected to another element/feature, and not necessarily mechanically. Likewise, unless expressly stated otherwise, “coupled” means that one element/feature is directly or indirectly coupled to another element/feature, and not necessarily mechanically, such as when elements or features are embodied in program code. Thus, although schematics shown in the figures depict example arrangements of processing elements, additional intervening elements, devices, features, components, or code may be present in an actual embodiment.

The invention may be described herein in terms of functional and/or block components and/or various processing steps. It should be appreciated that such block components may be realized by any number of hardware, software, and/or firmware components configured to perform the specified functions. For example, an embodiment may employ various integrated circuit components, e.g., memory elements, digital signal processing elements, logic elements, diodes, look-up tables, and the like, which may carry out a variety of functions under the control of one or more microprocessors or other control devices. Other embodiments may be employ program code, or code in combination with other circuit components.

In accordance with the practices of persons skilled in the art of computer programming, the present disclosure may be described herein with reference to symbolic representations of operations that may be performed by various computing components, modules, or devices. Such operations may be referred to as being computer-executed, computerized, software-implemented, or computer-implemented. It will be appreciated that operations that can be symbolically represented include the manipulation by the various microprocessor devices of electrical signals representing data bits at memory locations in the system memory, as well as other processing of signals. The memory locations where data bits are maintained are physical locations that have particular electrical, magnetic, optical, or organic properties corresponding to the data bits.

Unlike traditional sleep studies, the systems and methods described herein can achieve a level of comfort much closer to what users are accustomed to, making sleep analysis more practical and potentially more accurate and suitable for long-term data acquisition and analysis. The systems and methods have the ability to analyze a night's sleep in less than a minute, and thus, provide a economically efficient option to consumers who cannot currently afford a sleep study. As will be described, a user may upload their sleep data to a web service, where they can use a guided online interface with access to multiple analytics on sleep patterns. The user may also have access to an online sleep coach that can help develop healthy sleep habits.

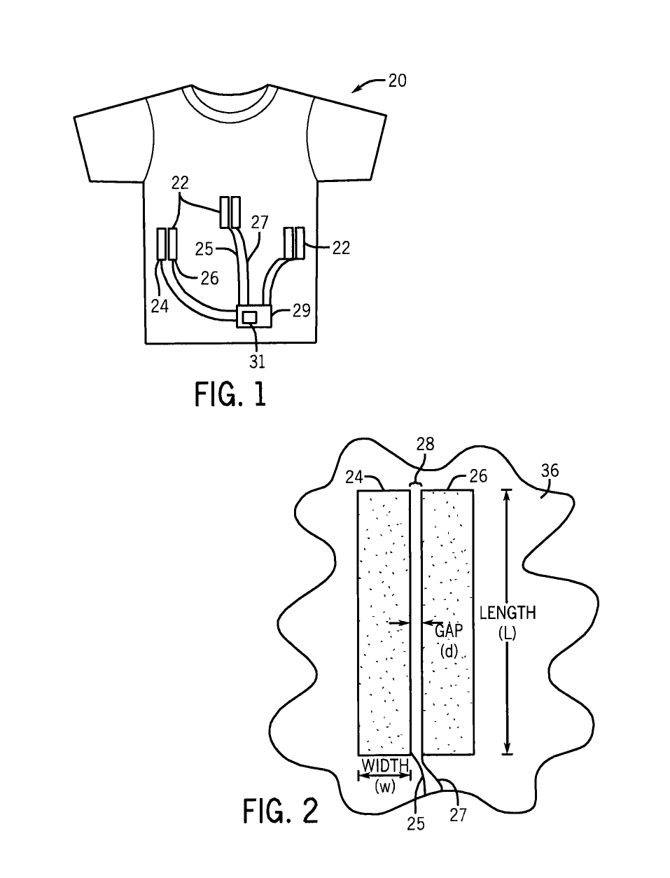

Turning now to the drawings, and referring initially to FIG. 1, a system 10 in accordance with the present invention includes an exemplary monitoring device 20 configured to be worn by a subject during a test period and a data processing system and user interface 21. As will be described in detail, the system 10 uses the basic principle that respiratory pattern changes with sleep stage, as well as with certain forms of sleep fragmentation, such as arousals and breathing disturbance. However, while substantial detail will be provided hereafter with respect to the clinical application of sleep monitoring, the systems described herein are readily applicable to other monitoring and analysis applications, including exercise and other extended states or for physiological event monitoring, such as to monitor coughing, sneezing, and the like.

The monitoring device 20 is preferably formed as an article of clothing, such as the illustrated shirt 23, and hereinafter will be referred to as shirt 23. However, it is to be appreciated that the shirt can take on other forms as well, including a long-sleeve shirt, a muscle shirt, a sleeveless shirt, or for example, simply a belt or a portion of fabric or other material having the electrical characteristics described below that is positioned on the subject during a test period. In embodiments utilizing the shirt 23 as a substrate for the monitoring device 20, the shirt 23 may be a form-fitting shirt, such as an UNDER ARMOUR® HEATGEAR® form-fitting shirt. Regardless of configuration, the monitoring device 20 as a data acquisition device that can be worn for days/weeks in the home environment. It represents an objective monitor of sleep quality and quantity.

Embodiments of the monitoring device 20 contain at least one embedded sensor 22 (three sensors are shown in the exemplary embodiment) configured to detect a characteristic of respiration of the subject over time. In some embodiments, the embedded sensors 22 serve as capacitors, such that chest expansion (movement) causes the sensors to move apart, which generates a continuous electrical signal representative of respiration. Using novel time-frequency analysis that may be performed by the data processing system 21 and will be discussed below, the patterns identified in the respiration can be used to quantify sleep stages and fragmentation.

Though described within the context of a substrate that is formed as clothing and, for example, a shirt, the sensor in accordance with the present invention was designed measure an amount of strain on an elastic substrate both locally and generally. Hence, the substrate need not be a shirt or even any clothing. Furthermore, the strain measured does not need to be connected to respiration or other physiological phenomenon. Referring to FIG. 2, the sensor design is composed of two main components. First, two areas or “pads” 24, 26, are arranged with a small gap 28 between the pads 24, 26. The pads 24, 26 have a low co-efficient of elasticity and are conductive. The two pads are each electrically conductive but disconnected to form a co-planer plate capacitor. The second component is an elastic substrate 36 to which the pads 24, 26 are attached. The substrate 36 allows the gap 28 to change with varying amounts strain on the elastic substrate.

The pads 24, 26 may be formed from a variety of conductive materials including conductive fabric, thread, metal films and plates, and conductive inks. The elastic substrate 36 can be formed of a variety of materials on which the conductive pads 24, 26 can be placed. Through testing it was shown that a pad 24, 26 with a rectangular shape and an approximate area of 5 square inches and a gap of several mm performed well in the application of physiological monitoring and, particularly, respiration monitoring. It was found that, in general, base capacitance was desirably between 10-20 pF and the capacitive change with strain was desirably on the order of 0-2 pF. Also, in the application of physiological monitoring and, particularly, respiration monitoring, it was found that, in general, single parallel lines or wires do not perform as well as the capacitive pads due to the sensing effect being caused by fringe capacitance. It was also found that, in general, pads made out of layers of conductive thread did not perform well.

This sensor design has several key features. First, the pads can be made of a variety of conductive materials. Many conductive materials are extremely thin and flexible making for a minimal hardware requirement and allowing the strain sensors to be flexible. Secondly, because a change in strain is being represented by a change in capacitance between the two pads, many readily available simple circuits can be used for detection. Because a change of capacitance can be recorded over a change of gap width of several mm, the range of strain that can be measured is several orders of magnitude greater than typical strain gauges.

Additionally, though the present description focuses on respiration monitoring and sleep monitoring therefrom, the sensors have broad applicability. For example, since most fabrics are elastic to a certain extent, the sensors can be placed on a wide variety of fabrics. Possible uses include detecting respiration through the strain induced by a moving thorax, movement detection of a body, limb, or joint by a change in localized strain, and deformations based on many localized sensors. More industrial uses have also been examined, such as detecting localized and general strain in ropes and rigging, parachutes, tents, and other strain critical fabrics. Because a fabric is not required, only an elastic substrate, many other non-fabric applications exist. Possible applications include diapers to measure respiration and movement in infants.

Referring again to FIG. 2, the pad 24 has an associated length 24L and width 24W, and pad 26 also has an associated length 26L and width 26W. In one embodiment, length 24L equals length 26L and width 24W equals width 26W.

The capacitance between the two pads 24, 26 is given by Equation 1:

$$C = ɛ L \frac{K ( \sqrt{1 - {k}^{2}} )}{K ( k )} ; \qquad Eqn . 1$$

where ∈ is permissivity, K(x) is the complete elliptic integral of the first kind, and kis given by Equation 2 below.

$$k = \frac{d}{2 w + d} . \qquad Eqn . 2$$

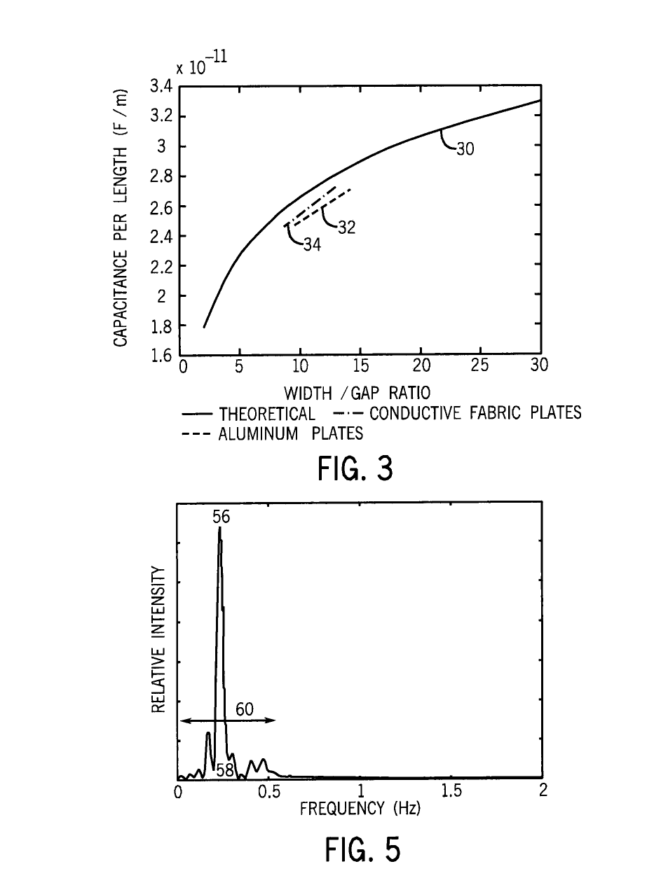

In order to validate the sensor model, the measured capacitance of two sets of pads, both in stretched configuration and un-stretched configuration, was compared with the theoretical results 30. FIG. 3 shows the comparison including aluminum pads 32, and conductive fabric pads 34. In addition to confirming the model, FIG. 3 shows that in a viable range of operation, the change in capacitance is approximately linear.

In one embodiment, each pad 24, 26 includes a conductive ink pad painted onto a substrate, which may then be laid directly onto the substrate 36 of the shirt 23. In addition to the conductive ink pad, a system for isolating the conductive portion of the pad may also be used to allow the system to be electrically isolated. This is achieved by first painting the conductive ink onto the pad, then placing a second pad on top of the first with overlapping edges, thereby sealing the conductive ink inside. These pads 24, 26 may then be embedded into the substrate 36 of the shirt 23 in a non-obstructive way to form the coplanar sensor 22, including pad 24 and pad 26, with the small gap 28 of elastic substrate 36 between them. The pads 24, 26 can be either embedded or sewn into the fabric.

As the subject's chest expands, the size of the gap 28 increases, causing a decrease in the capacitance between the two pads 24, 26. Accordingly, through a change in capacitance, the sensor 22 collects information related to a characteristic of respiration of the subject over time. More than one coplanar sensor 22 can be placed at intervals around the shirt 23 to ensure continuous respiration recording even when the subject shifts during the night and prevents a given pad 24, 26 or sensor 22 from operating. It is also important to identify that various subjects breath differently. Expansion of the abdomen and the lower chest can differ from subject to subject, and the placement of multiple sensors 22, such as the placement of the three sensors 22 in FIG. 1, for example, including a chest sensor and two side abdomen sensors, allows for all breathing patterns to be accommodated.

In some embodiments, to measure the change in capacitance between the two pads 24, 26, a signal from conductive threads 25 and 27 coupled to each pad 24, 26 respectively may be passed through collection circuitry 29. These connections may take various forms, including the use of magnet wire, thin wire, and conductive ink traces as routing members. Thus, the collection circuit 29 is connected to the sensor 22 to receive an indication of the characteristic of respiration detected by the sensor 22. The collection circuit may evaluate the sensor 22 capacitance against a known reference capacitor or other various mechanisms for evaluating the capacitance. This collection circuitry 29 may also convert a change in relative capacitance between the sensor 22 and the reference into a change in voltage, which can then be recorded and stored in memory as respiration data for wireless or later wired or wireless transmission to the processing system 21. Alternatively, the collection circuitry 29 may simply assemble the raw data from the sensors 22 and communicate the data, for example wirelessly or through a later downloading, to the processing system 21 for analysis. Whether processed by the collection circuitry 29 or the processing system 21, a time series of respiration data is yielded from the indication of the characteristic of respiration over time.

The collection circuitry 29 may be partially or fully removable when washing the shirt 23. In other cases, the shirt 23 may be designed for single use and not require disassembly for washing. The collection circuitry 29 can be configured to record multiple night's sleep (e.g., one, five, ten, or twenty or more) worth of data and can connect to a computer wirelessly and/or with the use of a wired connection, such as a universal serial bus (USB) connection, as non-limiting examples. A charger (not shown) may also be provided when the collection circuitry 29 is equipped with a rechargeable battery 31. In some embodiments, the data processing system 21 may be a personal computer and, once data is collected, the user can upload their data to the personal computer, or sign into a website 33, to view their sleep history and sleep health data, and use local or online tools to monitor and track their sleep wellbeing. The data can be processed by the collection circuitry 29, the data processing system 21, and/or the website or a server running the website 33, and may be output through multiple visual aids, including a sleep hypnogram and/or other analytics discussed below.

In some embodiments, a wireless connection with any of a variety of known signals, frequencies, and protocols may be used, including WiFi, 900 MHz, ZigBee, BLUETOOTH®, and the like, as non limiting examples, to stream live and/or stored respiration data to recording software that can display the data on the user interface of the data processing system 21 in real-time. Use of a wireless connection allows the subject to be monitored remotely without disrupting his or her sleep and for data to be stored remotely. In some embodiments, an automated method is able to quickly and accurately score a subject's sleep stages. For example, scoring may include creating a hypnogram and evaluating the hypnogram based on a threshold of multiple data points received from selected indices. The method may comprise a computer readable code embodied in or on a computer usable medium.

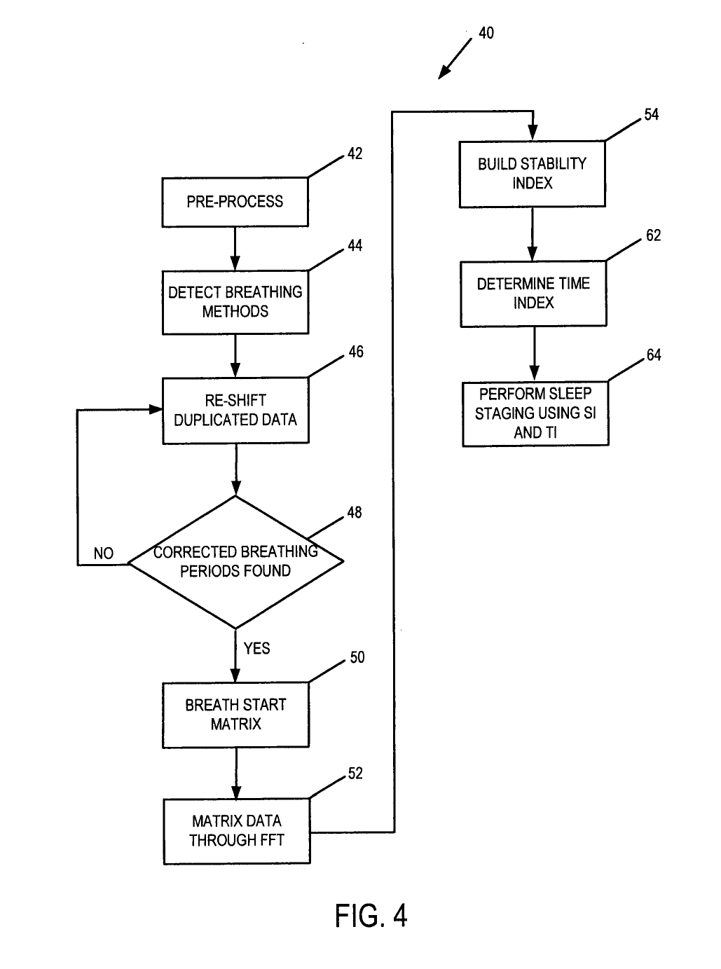

Referring now to FIG. 4, the steps of a method 40 that is consistent with embodiments described herein is illustrated. At process block 42, data acquisition begins and may include a pre-process step to represent the acquired respiration data as a function of amplitude versus time and then duplicate and time shift acquired amplitude versus time waveform. The initial time shift may be, for example, 0.5 seconds. Alternatively, the initial time shift may be selected such that, when the breaths are identified, the data is the shifted using half of the average breath duration. Furthermore, it is contemplated that a second shift can vary between 0 and the average breath duration.

The duplication and time shift provides a patient-specific mechanism for analysis that uses the detection of inherent characteristics and the frequencies thereof, rather than using abstract or predetermined thresholds or other non-patient specific metrics. Furthermore, because subjects of different sizes and different conditions will have varying amounts of chest expansion during respiration, it is desirable to provide an analysis tool that is independent of the magnitude of change in the chest expansion and the change in the capacitance experienced by the capacitive sensors due to chest expansion. Instead of analyzing amplitude changes of the chest, emphasis may be placed primarily on the frequency of its movement and any indicators exhibited by it.

Specifically, single breathing periods can be detected at process block 44 whenever the original respiration data set and the time-shifted respiration data set cross. Since typical respiration may exhibit sudden changes in “direction” whenever the chest is fully expanded or contracted, there can be small errors in “beat detections” if the data is not properly shifted. For example, it is contemplated that “changes in direction” may include changes in amplitude, changes in repeated inhalations, changes in frequency or phase (double breathing), or the like. “Beat detection” error refers to an occurrence when the data is shifted that causes the signals to cross at a point that does not correspond to the period of the breath. To correct for these possible errors, at process block 46, the duplicated respiration data may be re-shifted by the average period length, and the process of copying of the data, re-shifting, and detecting breaths is repeated once. The reason for the second shift or “re-shift” is that the first shift may be essentially random and small variations in the breathing can show up as full breaths. The “second shift” may be used to reiterate the process with a new data copy and a new shift at half the average breathing rate found from before. After finding the corrected breathing periods at decision block 48, a matrix is created at process block 50 indicating the start of each breath. The matrix is a method of storing start and stop points of each individual breath. If the corrected breathing periods are not found, the process of re-shifting the data is repeated at process block 46. By way of the foregoing exemplary process a patient or subject-specific mechanism for analysis is created that is not plagued by traditional methods that rely simply on abstract or predetermined thresholds that are not subject-specific and can be readily applied to different subjects of different sizes and or even the same subject that is subjected to different sleep conditions and will have varying amounts of chest expansion during respiration. That is, the acquired time series of data pertaining to respiration of the monitored subject is a process to identify a subject-specific mechanism for identifying inhalation or exhalation.

To build an accurate model of breathing volatility over time, the method 40 uses a novel time-frequency analysis by running small samples of this raw respiration data through a fast Fourier transform (FFT) at process block 52. Small samples may refer to data from a small number of breaths, for example, 3-5 breaths, with breaths indicated by time between crosses. A desirable respiration window size was found, through testing, to be about 3-5 breathing periods, although more or less may be used. Each individual FFT yields three data points that are used to create a Stability Index (SI) for the optimal respiration window, as indicated at process block 54. The SI is, extending the example used above with respect to the initial matrix, a method of storing peak frequency, peak intensity, and peak bandwidth for each of the breathing windows analyzed by the FFT.

Referring to FIG. 5, one of the data points from the FFT is the frequency with the largest intensity, shown as point 58, and is the dominant respiration frequency in the FFT. The relative intensity of the dominant frequency is shown as point 56, and represents the periodic nature of the breathing in this respiration window. Lastly the width, or bandwidth, of the dominant frequency peak, shown by arrow 60, acts as a third indicator of stability. Because the frequency peak of the FFT is typically at a very low frequency, the run time of the algorithm analyzing the respiration data is decreased by simply setting the bandwidth equal to the frequency corresponding to 0.5% of the peak intensity 56. Thus, point 56 is the relative intensity (top of the peak), point 58 is the dominant frequency, and point 58 is the frequency with max relative intensity.

It has been found that an instantaneous categorization of a respiration window alone may not be satisfactory for determining its corresponding sleep stage in some settings. As a result, the SI may not be solely used for staging. Referring again to FIG. 4, at process block 62, a Time Index (TI) may then be created based on a relative change in the SI over various windows. In some embodiments, the method collects five consecutive SI samples and determines the mean change between them to determine the TI, although more or less consecutive SI samples may be used. Like the SI and extending the example used above with respect to the initial matrix, the TI is a method of storing average rate of change of each peak frequency, peak intensity, and peak bandwidth for each of the breathing windows analyzed bu the FFT. This TI represents how quickly the breathing frequency is changing, the stability of the dominant frequency, and the time-variance of the bandwidth, as described above with respect to FIG. 5. In some embodiments, the method may use both the SI and TI to determine a respiration window's corresponding sleep stage in order to produce an accurate sleep staging hypnogram, as indicated at process block 64. Such a hypnogram or other report can be provided on the sleep of the subject over time. In this regard, the stability index and the time index and, thereby a determination and tracking of sleep state may be generated based only on the characteristic of respiration of the subject over time and information derived therefrom.

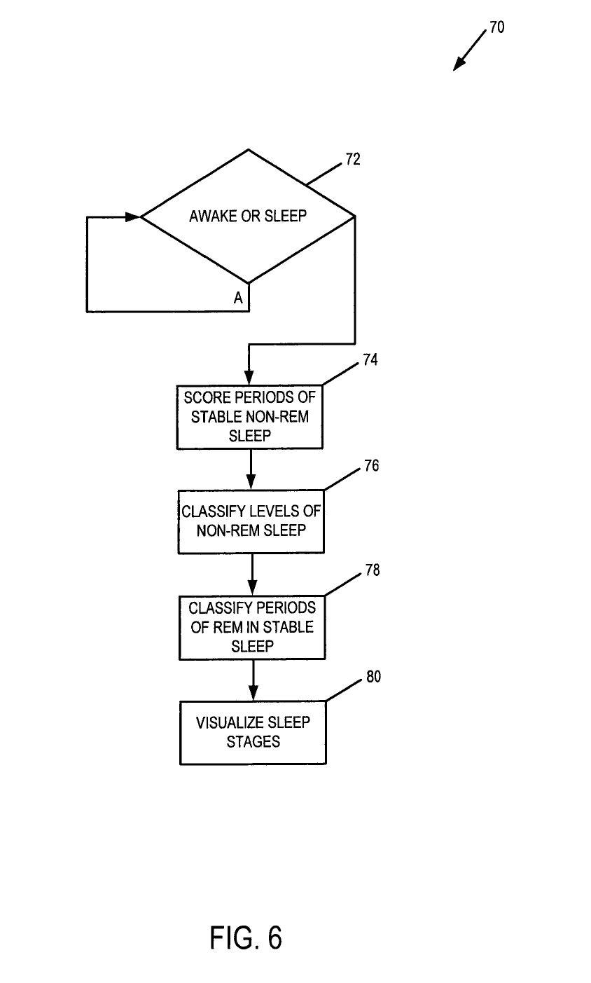

Referring to FIG. 6, the steps of a sleep staging method 70 are described in greater detail. In some embodiments, the method 70 first determines if the subject is asleep by checking for both unstable breathing and rapidly changing intensity of the dominant frequency, as indicated at decision block 72. These areas of wakefulness are determined when the breathing stability's magnitude is less than its change. By subtracting the TI from the SI, it is possible to quickly determine awake periods, for example, using a negative threshold.

Next, at process block 74, the method may score or classify periods when the subject was in stable, non-REM sleep. It was found that during this stable stage, the intensity of the dominant respiration frequency 56 maintains a large, steady value. In addition, both the time-averaged change in intensity and the dominant respiration frequency drop. By setting thresholds, as may be selected by a user or through experimental determination, based on the mean difference between the SI and the TI, as well as the average respiration rate found after the first data shift and then recalculated to a more exact value with the second data shift, it is possible to reliably find periods of stable, non-REM sleep. At process block 76, the method may optionally further classify the various levels of non-REM sleep by comparing the relative level of intensity of the dominant respiration frequency.

The method 70 next finds periods of REM in periods of stable sleep, as indicated at process block 78. To accomplish this, the method looks for periods when the time-averaged change of the respiration is greater than a predetermined threshold. These periods represent stable respiration with a rapidly changing frequency. In some embodiments, the method may only look for REM within periods of stable sleep and may not recognize periods where a subject drops between wake and REM quickly. In other embodiments, this can be resolved by creating a REM threshold that includes periods of wake as opposed to only stable sleep periods.

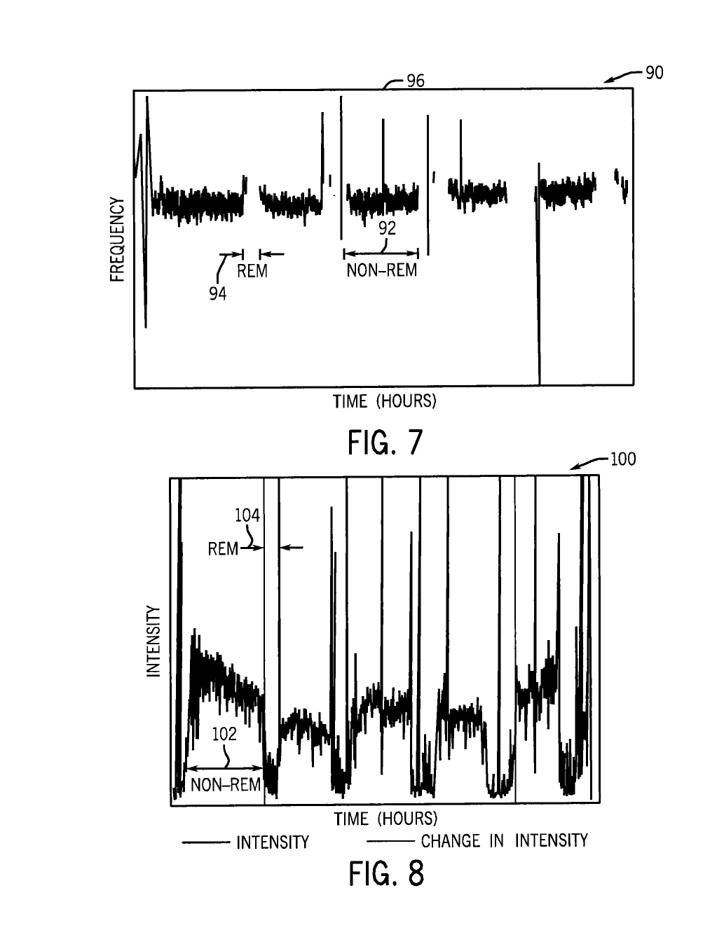

An optional method step includes visualization of the patient's sleep stages, as indicated at process block 80. Besides the standard sleep staging hypnogram that is commonly used, embodiments of the invention may provide visual tools to help visually categorize a patient's sleep. FIG. 7 shows a sleep heat map 90 that may be generated directly from the FFTs. The image shows frequency over time with intensity indicated by color. Stable non-REM sleep periods may be indicated by bright areas 92 on the heat map 90 representing strong, stable respiration. REM periods, indicated by gaps of darker blue 94 when the respiration frequency becomes more variable. Awake periods stand out as taller frequency bands 96 with a darker red color as the respiration exhibits more randomness.

FIG. 8 shows an additional visual aid consisting of a frequency stability graph 100. The frequency stability graph 100 may help to further analyze the relative depth of sleep. Shown on the graph 100 are both dominant frequency intensity and time averaged change in intensity versus time. Periods of non-REM, stable sleep show up as a rise in dominant frequency intensity identified with arrow 102, and REM stages can be categorized when the relative intensity drops to the level of the change of intensity, identified with arrow 104.

### Example

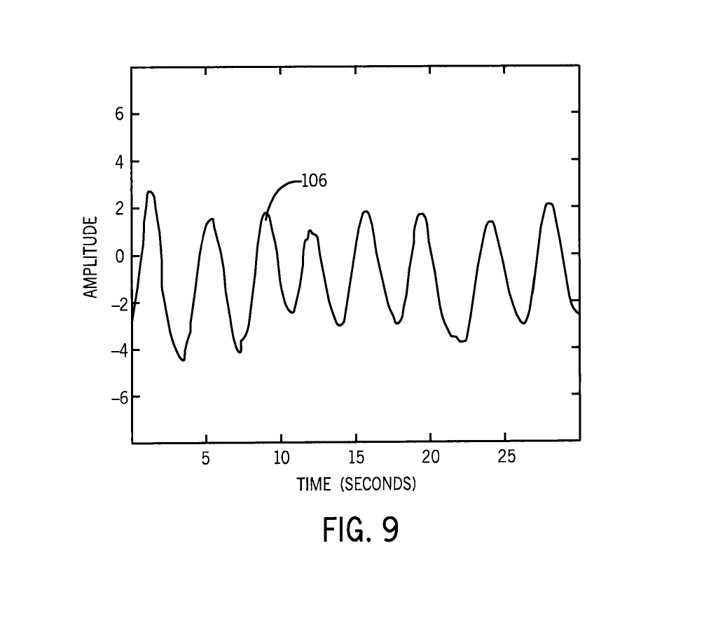

In order to validate the system's ability to accurately detect a clean respiration signal, a prototype test was conducted in which a subject was asked to sleep while being monitored both with the above-described system, including shirt 23 and associated sensors 22 of FIGS. 1 and 2 and the traditional (e.g., PSG) sleep study sensors. The test subject consisted of a healthy 21-year-old male with no known sleep disorders. The shirt 23 was connected to a Bluetooth device that transmitted data to an adjacent room during the test. This allowed for continuous monitoring of the data. A data logger (not shown) in parallel maintained a copy of the respiration data, and, after over two hours of continuous data collection, the subject was awoken. Shown in FIG. 9 is a sample of the respiration data gathered from the shirt 23.

The shirt 23 was able to gather a clean respiration signal 106 from the subject. With a small amount of post processing, such as removing high-frequency noise, a respiration signal that was nearly identical to the respiration trace from a standard respiration belt from the sleep lab was achieved. While the measured amplitude was smaller than the data from a standard inductive belt, the relative magnitudes were the same and the difference can easily be adjusted with an additional amplifier stage(s).

To validate the sleep scoring method, data from existing sleep studies was gathered. The data was parsed and only a single respiration channel was used in addition to a technicians scoring of each study. The method was tested with each study and the output hypnogram was compared to that of the technicians by percentage time spent in both non-REM and REM stages of sleep. The method correctly scored each study with approximately 70 percent to 80 percent accuracy, which is comparable to that of a technician, while having the advantage of being completely repeatable and, if desired, fully automated.

This written description uses examples to disclose the invention, including the best mode, and also to enable any person skilled in the art to practice the invention, including making and using any devices or systems and performing any incorporated methods. The patentable scope of the invention is defined by the claims and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

Finally, it is expressly contemplated that any of the processes or steps described herein may be combined, eliminated, or reordered. Accordingly, this description is meant to be taken only by way of example, and not to otherwise limit the scope of this invention.

1-22. (canceled)

23. A system configured to monitor sleep of a subject, the system comprising:
a garment forming substrate having a elasticity;
a first conductive pad engaged with the substrate and extending along a length and a width to form a plane;
a second conductive pad engaged with the substrate, extending along a length and a width to form a plane, and arranged proximate to the first conductive plane;
a dielectric gap formed between the first and second conductive pads such that at least one of a length and a width of the first conductive pad extends substantially in parallel to at least one of a length and a width of the second conductive pad;
a processor coupled to the first conductive pad and the second conductive pad and configured to monitor a relative capacitance between the first conductive pad and the second conductive pad as the dielectric gap is varied in response to movement imparted to the substrate and generate an indication of a characteristic of the movement imparted to the substrate.

24. The system of claim 23 wherein the processor is further configured to translate the characteristic of the movement imparted to the substrate into an indication of respiration.

25. The system of claim 23 wherein the first pad and the second pad are formed from at least one of conductive fabric, thread, metal film, and conductive ink.

26. A system configured to monitor sleep of a subject, the system comprising:
a elastic substrate configured to be mounted to the subject;
a sensor mounted on the elastic substrate and configured to determine movement of the subject when the elastic substrate is mounted to the subject to communicate determined movements as a feedback signal;
a communications circuit configured to receive the feedback signal and transmit the feedback signal as one of a live stream and a stored signal;
a data processing system configured to receive the one of the live stream and the stored signal;
a display configured to provide a user interface providing feedback regarding the one of the live stream and the stored signal in real-time.

27. The system of claim 26 wherein the data processing system is configured to derive sleep information about the subject from the one of the live stream and the stored stream.

28. The system of claim 26 wherein the user interface includes a hypnogram derived from the sensor data.

29. The system of claim 26 wherein the data processing system is configured to determine frequency information over time about determined movements from the one of the live stream and the stored stream.

30. The system of claim 29 wherein the display is configured to illustrate the frequency information over time with intensity indicated by color.

31. The system of claim 26 wherein the data processing system is configured to detect respiration from the one of the live stream and the stored stream by determining an electrical signal strain induced by stretching of the elastic substrate.

32. The system of claim 26 wherein data processing system is configured to detect at least one of a moving thorax, a movement detection of a body limb, and a movement of a joint by determining at least one of a change in localized movement and deformations based on many localized movements determined by the sensor and represented in the one of the live stream and the stored stream.

33. A system configured to monitor sleep of a subject, the system comprising:
a substrate configured to be worn by the subject;
a sensor mounted on the substrate and configured to determine movement of the subject when the substrate is mounted to the subject communicate determined movements as a feedback signal;
a memory configured to receive the feedback signal and store the feedback signal as a series of data;
at least one of a wireless and a wired communications circuit configured to communicate the series of data; and
a data processing system configured to receive the series of data and analyze the series of data to determine a respiratory pattern of the subject from the series of data and changes in the reparatory pattern indicative of sleep fragmentation.

34. The system of claim 33 wherein the data processing system is further configured to determine at least one of arousals and breathing disturbances from the series of data.

35. The system of claim 33 wherein the data processing system is further configured to monitor at least one of coughing and sneezing using the series of data.

36. The system of claim 33 wherein the sensor, memory, and at least one of a wireless and a wired communications circuit are configured to be removable from the substrate.

37. The system of claim 33 wherein the substrate forms clothing.

38. A method for tracking at least one of respiration in an infant comprising the steps of:
using wearable monitoring system that includes at least one sensor, detecting a characteristic of respiration of the infant over time;
processing the characteristic of respiration in the infant over time to identify breathing periods of the infant;
creating a stability index from data points within the breathing periods;
creating a time index based on a relative change in the stability index over time;
generating a report about the infant over time; and
wirelessly communicating at least one of the characteristic of respiration of the infant, the stability index, the time index, and the report, to be displayed on a display with a user interface.

39. The method of claim 38 further comprising identifying periods when a time-averaged change of the respiration is greater than a predetermined threshold.

40. The method of claim 38 further comprising determining at least one of coughing and sneezing from the characteristic of respiration of the infant over time.

41. The method of claim 38 further comprising, using the wearable monitoring system that includes the at least one sensor, detecting a movement of the infant over time.

42. The method of claim 38 wherein the wearable monitoring system includes a shirt and the sensor is formed a capacitive sensor mounted to the shirt.

43. A system for collecting and analyzing sleep of a subject, the system comprising:
a sensor configured to detect a characteristic of respiration of the subject;
a collection circuit connected to the sensor to receive an indication of the characteristic of respiration detected by the sensor;
a processing system configured to:
communicate with the collection circuit and generate a time series of respiration data from the indication of the characteristic of respiration over time;
process the time series of respiration data to identify a subject-specific mechanism for identifying at least one of inhalation and exhalation;
analyze respiration volatility using the time series of respiration data and subject-specific mechanism for identifying at least one of inhalation and exhalation;
using the respiration volatility, generate a stability index relating fundamental characteristics of the time series of respiration data to metrics of stability; and
based on the stability index perform a sleep staging analysis to generate a report on the sleep of the subject over time.

44. The system of claim 43 wherein the processing system is further configured to analyze the respiration volatility by performing a fast Fourier transform (FFT) on the time series of respiration data.

45. The system of claim 43 the stability index includes at least one of a frequency within the time series of respiration data with a largest intensity and a dominant respiration frequency within the time series of respiration data.

46. The system of claim 43 wherein the stability index includes at least one of a relative intensity of a dominant frequency within the time series of respiration data and a bandwidth of the dominant frequency peak within the time series of respiration data.

47. The system of claim 43 wherein the processing system is further configured to generate a time index using the stability index by determining a relative change in the stability index over a given time period.

48. The system of claim 47 wherein the processing system is further configured to generate the time index as a representation of at least one of a rate of change in breathing frequency, a stability of a dominant frequency in the time series of respiration data, and a time-variance of a bandwidth of the dominant frequency peak within the time series of respiration data.

49. The system of claim 47 wherein the processing system is further configured to collect a given number of consecutive stability index samples and determine a mean change between the given number of consecutive stability index samples to determine the time index.

50. The system of claim 43 wherein the sensor includes a capacitive sensor configured to detect the characteristics of respiration of the subject based on changes in capacitance of the capacitive sensor induced by respiration of the subject.

51. A system for performing sleep staging of a subject, the system comprising:
at least one capacitive sensor that provides sensor capacitance data configured to vary over time based on breathing periods of the subject;
a circuit configured to evaluate the sensor capacitance data against a known reference capacitor; and
a computer-readable code embodied in or on a computer usable medium, the computer readable code configured to cause a computer to perform the steps of:
processing the capacitance data to identify breathing periods of the subject;
creating a matrix that indicates the start of each breath;
running the matrix data through at least one fast Fourier transform;
creating a stability index from data points generated by the at least one fast Fourier transform;
creating a time index based on a relative change in the stability index over time; and
determining sleep stages using the stability index and the time index.

52. The system according to claim 51 wherein the capacitive sensor comprises at least two coplanar pads with a small gap between the pads.
